(19) United States
(12) Patent Application Publication
(10) Pub. No.: US 2018/0200220 A1
(43) Pub. Date: Jul. 19, 2018

(54) PROPHYLAXIS AND MITIGATION OF MIGRAINE HEADACHES USING KETOGENIC MEDIUM CHAIN TRIGLYCERIDES, KETONE ESTERS, AND OTHER KETOGENIC SOURCES

(71) Applicant: COGNATE NUTRITIONALS, INC., Bloomfiield, CT (US)

(72) Inventors: Robert A Firger, Bloomfield, CT (US); Theodore B VanItallie, Old Lyme, CT (US)

(21) Appl. No.: 15/743,448

(22) PCT Filed: Jul. 8, 2016
(86) PCT No.: PCT/US16/41474

§ 371 Date: Jan. 10, 2018

Related U.S. Application Data
(60) Provisional application No. 62/191,075, filed on Jul. 10, 2015.

(51) Int. Cl. A61K 31/23; A61P 25/06; A61K 36/889

(52) U.S. Cl. CPC A61K 31/23; A61K 36/889; A61P 25/06

(57) ABSTRACT

The invention provides methods of reducing the frequency of migraine onset in a migraineur, reducing migraine symptoms experienced by a migraineur, augmenting the effects of pharmaceutical intervention to reduce the frequency of migraine onset in a migraineur, augmenting the effects of pharmaceutical intervention to reduce the migraine symptoms experienced by a migraineur, augmenting the effects of non-pharmaceutical intervention to reduce the frequency of migraine onset in a migraineur, and/or augmenting the effects of on-pharmaceutical intervention to reduce migraine symptoms experienced by a migraineur. The methods according to the invention comprise providing the migraineur with dietary KMCT.

### BACKGROUND OF THE INVENTION

### Field of the invention

The invention relates to migraine headaches, sometimes simply called migraine. More particularly, the invention relates to preventing, arresting, or reducing the frequency or severity of migraine.

### SUMMARY OF RELATED ART

Migraine is a neurological disease with a strong genetic component. It is characterized by episodes of disabling headache, often called migraine attacks. They are clinically quite different from regular headaches which are non-migrainous. There are about 100 million people with headaches in the U.S.; about 37 million of these people have migraines. The World Health Organization estimates that approximately 18 percent of women and 7 percent of men in the U.S. suffer from migraines. People who suffer from migraine are known as “migraineurs”. Migraine ranks in the top 20 of the world's most disabling medical illnesses. (See Global Burden of Disease Study, updated 2004, World Health Organization)

Migraines are called primary headaches because the pain isn't caused by another disorder or disease such as a brain tumor or head injury. Some cause pain on just the right side or left side of the head (hemicrania), others result in generalized head pain. Migraine sufferers may have moderate or severe pain and usually can't participate in normal activities, during the duration of the attack, because of the pain. Often when a migraine strikes, people can't think beyond trying to find a quiet, dark room. More than 90% of sufferers are unable to function normally during their migraine attacks. Because of migraine-induced disability, American employers lose more than $13 billion each year as a result of 113 million lost work days.

Many people experience migraine attacks lasting for at least four hours and which may last for days. The range of time someone is affected by an attack is actually longer than the full-blown attack itself, as there is a pre-monitory, or build-up phase, and a post-dromal phase that can last one to two days. An estimated 14 million individuals in the U.S. are classified as “chronic migraneurs”, meaning that they suffer a migraine episode a minimum of 15 days each month. For all intents and purposes, this means that with migraine symptoms usually lasting multiple days, these sufferers may experience the impact of migraine on their lives virtually every day.

Recent research has highlighted the potential importance of neurogenic inflammation in migraine pathophysiology and pharmacology. Extravasation (inflammatory leakage of white blood cells from the capillaries), vasodilatation (widening of blood vessels resulting in lowered blood pressure), mast cell activation, and the release of pro-inflammatory mediators may activate trigeminal afferents (the nerve fibers of the fifth cranial nerve governing sensation in the face and certain facial motor activity), thus leading to sensitization in the migraineur. There are indications that blockading vasodilatation in inflammation may be effective in the treatment of migraine. It has been shown that the ketone body β-hydroxybutyrate has beneficial effects on inflammation, with significant potential for addressing aspects of a variety of metabolic related diseases.

Other applications of the uses of KMCT and ketones to address inflammatory conditions, for example in the field of Traumatic Brain Injury, are disclosed in co-pending U.S. application Ser. No. 62/030,285.

### Current Treatments of Migraine

A number of different medications have been used to treat migraine, with varying levels of effectiveness. All of these treatments, however, with increased frequency of use can lead to a condition called medication overuse headache (MOH). Medication-overuse headaches occur when medications not only stop relieving pain but themselves also cause headaches. Patients then use more pain medication, which may lengthen the duration of pain of the migraine sufferer. In addition, these treatments pose additional, specific risks such as drug sensitivities, cognitive symptoms and negative physiological effects, some of which are further elucidated below.

For pain associated with mild or moderate migraine, aspirin or non-steroidal anti-inflammatory drugs (NSAIDs, such as ibuprofen and naproxofen) are commonly used. Unfortunately, regular use of these medications can result in abdominal pain and intestinal ulcers—often associated with hemorrhage, and they are generally not effective against more severe migraine. Front-line treatments for more severe migraine include the triptans, such as sumatriptan (Imitrex), rizatriptan (Maxalt), almotriptan (Axert), naratriptan (Amerge), zolmitriptan (Zomig), frovatriptan (Frova) and eletriptan (Relpax). In addition to MOH, these medications can cause a condition called “serotonin syndrome” when used together with selective serotonin reuptake inhibitors (SSRIs), including Prozac, Celexa, Luvox, Zoloft, Paxil and Lexapro; or serotonin and norepinephrine inhibitors (SNRIs), including Effexor, Pristiq and Cymbalta , all of which are commonly prescribed for depression and/or anxiety. These are mental health conditions for which migraneurs already may be receiving treatment with some frequency. The front-line medications, discussed above, are often serotonin agonists whose nature it is to raise serotonin levels. When taken in combination, they can cause serotonin levels in the CNS (central nervous system) to rise to especially high levels. “Serotonin syndrome” is a rare, potentially life-threatening condition that occurs when an excess of serotonin—a neurotransmitter of major importance in the central nervous system (CNS) accumulates in the brain.

Less effective than triptans are ergotamines, such as ergotamine and dihydroergotamine. Ergotamines appear to be most useful for people who have migraines that last a long period of time, but don't have frequent migraine attacks. Side effects of ergotamines include dizziness, nausea and vomiting, cold, clammy hands and feet, muscle pain, numbness, and feelings of discomfort or anxiety. Ergotamines also can have serious interactions with a large number of drugs.

Medications containing narcotics, particularly codeine, are sometimes used to treat migraine headache pain for people who can't take triptans or ergotamines. Narcotics are seriously habit-forming and are usually used only as a last resort.

Glucocorticoids, such as prednisone and dexamethasone may be used in conjunction with other medications to improve pain relief. Because of the risk of adverse side effects, glucocorticoids should not be used frequently or for prolonged periods, which presents problems for chronic migraneurs.

Due to the severity and frequency of the side effects caused by many of these medications that treat symptoms of migraine, another strategy is to utilize medications that reduce the frequency of migraine attacks.

One front line treatment for reducing frequency of migraine onsets is beta blockers, which are normally and usually prescribed for high blood pressure and heart problems. Unfortunately, beta blockers produce their own, often severe side effects, including fatigue, nausea, reduced ability to exercise, insomnia, sleep problems, nightmares and vivid dreams, memory problems, depression, weight gain, and exacerbation of asthma.

Calcium channel blockers, which are also normally prescribed for high blood pressure and heart problems, are sometimes prescribed to reduce frequency of migraine onset. However, these drugs carry their own side effect profile, including dizziness, drowsiness, constipation, nausea, headache, rash, pitting edema of the feet, ankles and lower legs (owing to water retention), low blood pressure, tachycardia (rapid heartbeat), and flushing (reddening) of the face, neck and/or upper chest.

Tricyclic antidepressants are sometimes used to reduce frequency of migraine onset. Unfortunately, these drugs have a wide range of undesirable side effects, including disturbances in heart rhythm, increased sensitivity to sunlight, drowsiness, dry mouth, painful urination, sexual dysfunction, weight gain, dizziness, lightheadedness, and headaches. In addition, antidepressants come with the U.S. Food and Drug Administration's strongest “black box” warning that the use of antidepressants has been associated with an increased risk of suicidal thoughts and behaviors in children, adolescents and young adults. Tricyclic antidepressants can be fatal in overdose.

Anticonvulsants are sometimes prescribed to reduce frequency of migraine onset. Only Depakote (gen. valproic acid) and Topamax (gen. topiramate) have been approved for this purpose. However, some anticonvulsant medications may reduce the effectiveness of oral contraceptives, since their induction of the hepatic cytochrome P450 (CYP450) isoenzyme causes decreased sex hormone levels in women taking oral contraceptives, thus raising the potential for decreased effectiveness of oral contraceptives and increased risk of unplanned pregnancy. In addition, more than half of people who take anticonvulsant drugs report experiencing at least one side effect, the most common of which are dizziness, nausea and sleepiness. Some people who take newer drugs in this class also experience swelling in the feet and hands, weight gain, blurry vision, trouble concentrating, and lapses in memory.

More recently, injections of botulinum toxin (Botox®) have been used to reduce frequency of migraine onset. However, Botox's side effects include headache, facial loss of movement, eyelid drooping, lung inflammation, neck pain, muscle stiffness and weakness, muscle pain and spasms, pain at injection site, and high blood pressure. In addition, the paralyzing effect of Botox can spread to other areas of the body and can cause general weakness, double vision, difficulty swallowing, voice and speech disorders, loss of bladder control and difficulty breathing. Given the frequency and severity of side effects of both medications to treat symptoms of migraine and to reduce frequency of migraine onset, there clearly is a need for safer treatments to prevent or reduce frequency of migraine onset and reduce migraine symptoms. Although not yet a medically accepted treatment for migraine, some references have suggested the use of a ketogenic diet for this purpose (see Maggioni, F. et al. Cephalalgia 31, 1150-1). However, some authors have described ketogenic dieting as potentially causing severe headaches, and low glucose levels, among a number of other physiological effects of ketogenic dieting, which might complicate its use. In addition, adherence to a ketogenic diet can be difficult and compliance problems frequently arise.

### BRIEF SUMMARY OF THE INVENTION

The invention provides effective, safer treatments to prevent or reduce frequency of migraine onset and reduce migraine symptoms. The present inventors have surprisingly discovered that providing ketogenic medium chain triglycerides (KMCT) to patients suffering from frequent migraine attacks can reduce frequency of migraine onset and reduce migraine symptoms, without the side effects of medications currently used for this purpose. KMCT are metabolized in the liver to provide a rich source of ketone bodies, which can be metabolized as a carbon and energy source for the body, especially the brain. Unlike ketogenic dieting, providing exogenous KMCT does not result in reduced glucose concentrations or other physiological effects associated with ketogenic dieting, and does not suffer from similar patient compliance issues. Ingestion of KMCT has no reported serious side effects and only minor and transitory reported effects of gastro-intestinal distress or sensitivity in some users, which has been shown to usually diminish with continued use.

In a first aspect the invention provides a method for preventing or reducing the frequency of migraine onset in a migraineur. The method according to this aspect of the invention comprises providing the migraineur with dietary KMCT.

In a second aspect, the invention provides a method for reducing the migraine symptoms experienced by a migraineur. The method according to this aspect of the invention comprises providing the migraineur with dietary KMCT.

In a third aspect, the invention provides a method for augmenting the effects of pharmaceutical intervention to reduce the frequency of migraine onset in a migraineur. The method according to this aspect of the invention comprises providing the migraineur with dietary KMCT in combination with a pharmaceutical agent provided to reduce the frequency of migraine onset in a migraineur.

In a fourth aspect, the invention provides a method for augmenting the effects of pharmaceutical intervention to reduce the migraine symptoms experienced by a migraineur. The method according to this aspect of the invention comprises providing the migraineur with dietary KMCT in combination with a pharmaceutical agent provided to reduce migraine symtoms experienced by a migraineur.

In a fifth aspect, the invention provides a method for augmenting the effects of non-pharmaceutical intervention to reduce the frequency of migraine onset in a migraineur. The method according to this aspect of the invention comprises providing the migraineur with dietary KMCT in combination with a non-pharmaceutical intervention or agent.

In a sixth aspect, the invention provides a method for augmenting the effects of non-pharmaceutical intervention to reduce migraine symptoms experienced by a migraineur. The method according to this aspect of the invention comprises providing the migraineur with dietary KMCT in combination with a non-pharmaceutical intervention or agent.

In a seventh aspect, either separately or in combination with any of the six aspects above, the invention provides a method for preventing or reducing the frequency of onset of migraine, and/or reducing migraine symptoms, comprising providing a migraineur with dietary ketone esters, ketone salts and other sources of ketone bodies.

### DETAILED DESCRIPTION OF THE PREFERRED EMBODIMENTS

The invention relates to migraine headaches, sometimes simply called migraine. More particularly, the invention relates to preventing or reducing the frequency of migraine onset and reducing migraine symptoms. The invention provides effective, safer treatments to reduce frequency of migraine onset. The present inventors have surprisingly discovered that providing ketogenic medium chain triglycerides (KMCT) to patients suffering from frequent migraine attacks can reduce frequency of migraine onset and reduce migraine symptoms, without the side effects of medications currently used for this purpose. KMCT are metabolized in the liver to provide a rich source of ketone bodies, which can be metabolized as a carbon and energy source for the body, especially the brain. Unlike ketogenic dieting, providing exogenous KMCT does not result in reduced glucose concentrations or other physiological effects associated with ketogenic dieting, and does not suffer from similar patient compliance issues.

In a first aspect the invention provides a method for preventing or reducing the frequency of migraine onset in a migraineur. The method according to this aspect of the invention comprises providing the migraineur with dietary KMCT. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 3 g to about 15 g/day.

In a second aspect, the invention provides a method for reducing the migraine symptoms experienced by a migraineur. The method according to this aspect of the invention comprises providing the migraineur with dietary KMCT. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 10 g to about 30 g/day.

A convenient source for such KMCT is Fuel for Thought® (Cognate Nutritionals, Inc., Bloomfield, Conn.) an orally bioavailable nutritional supplement comprising capric and caprylic triglycerides and coconut oil. Other sources of KMCT, for example, are disclosed in co-pending U.S. applications Ser. Nos. 62/151,678 and 62/151,691.

In a third aspect, the invention provides a method for augmenting the effects of pharmaceutical intervention to reduce the frequency of migraine onset in a migraineur. The method according to this aspect of the invention comprises providing the migraineur with dietary KMCT in combination with a pharmaceutical agent provided to reduce the frequency of migraine onset in a migraineur. Such augmentation of pharmaceutical intervention may reduce the dosage and/or frequency of pharmaceutical agent required, thereby reducing unwanted side effects caused by the pharmaceutical agent. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 3 g to about 15 g/day. Pharmaceutical agents useful in this aspect of the invention include, without limitation, one or more of beta blockers, calcium channel blockers, tricyclic antidepressants, anticonvulsants (such as Depakote and Topamax), and Botox.

In a fourth aspect, the invention provides a method for augmenting the effects of pharmaceutical intervention to reduce the migraine symptoms experienced by a migraineur. The method according to this aspect of the invention comprises providing the migraineur with dietary KMCT in combination with a pharmaceutical agent provided to reduce migraine symptoms experienced by a migraineur. Such augmentation of pharmaceutical intervention may reduce the dosage and/or frequency of pharmaceutical agent required, thereby reducing unwanted side effects caused by the pharmaceutical agent. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 10 g to about 30 g/day. Pharmaceutical agents useful in this aspect of the invention include, without limitation, one or more of aspirin, acetominaphen, non-steroidal anti-inflammatory drugs (NSAIDs, such as ibuprofen and naproxofen), triptans (such as sumatriptan (Imitrex), rizatriptan (Maxalt), almotriptan (Axert), naratriptan (Amerge), zolmitriptan (Zomig), frovatriptan (Frova) and eletriptan (Relpax)), ergotamines (such as Ergotamine and Dihydroergotamine), opioid medications, and glucocorticoids (such as prednisone and dexamethasone).

In a fifth aspect, the invention provides a method for augmenting the effects of non-pharmaceutical intervention to reduce the frequency of migraine onset in a migraineur. The method according to this aspect of the invention comprises providing the migraineur with dietary KMCT in combination with a non-pharmaceutical intervention or agent. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 3 g to about 15 g/day. Non-pharmaceutical interventions and agents include, without limitation, one or more of transcutaneous electrical nerve stimulation, transcranial magnetic stimulation, biofeedback, acupuncture, cognitive behavior therapy, ketogenic dieting, butterbur extracts, feverfew, L-cysteine, riboflavin, coenzyme-Q supplements, magnesium supplements, tocopherols (vitamin E), calciferols (vitamin D), ascorbic acids and omega fatty acids.

In a sixth aspect, the invention provides a method for augmenting the effects of non-pharmaceutical intervention to reduce migraine symptoms experienced by a migraineur. The method according to this aspect of the invention comprises providing the migraineur with dietary KMCT in combination with a non-pharmaceutical intervention or agent. In some embodiments, the KMCT comprise a mixture of capric and caprylic triglycerides. In some embodiments the KMCT are enriched or purified from coconut oil. In some embodiments the KMCT comprise a mixture of capric and caprylic triglycerides and coconut oil. In some embodiments, the total KMCT provided to the migraineur is from about 3 g to about 30 g/day. In some embodiments, the total KMCT provided to the migraineur is from about 10 g to about 30 g/day. Non-pharmaceutical interventions and agents include, without limitation, one or more of caffeine, melatonin, magnesium, feverfew, butterbur, willow extract, ginger, valerian, coriander seed, dong quai root, rosemary, linden, honeysuckle, mullien, yarrow, wintergreen evadia, tocopherols (vitamin E), calciferols (vitamin D), ascorbic acids and omega fatty acids.

In a seventh aspect, either separately or in combination with any of the six aspects above, the invention provides a method for preventing or reducing the frequency of onset of migraine, and/or reducing migraine symptoms, comprising providing a migraineur with dietary ketone esters, ketone salts or other sources of ketone bodies. Preferably the ketone esters or ketone salts are provided in an amount to raise plasma levels of ketone bodies (acetoacetate and β-hydroxybutyrate) to from about 2 mM to about 5 mM. Preferred ketone esters and ketone salts include, without limitation, β-hydroxybutyrate-1,3-butanediol monoester, glyceryl-tris-3-hydroxybutyrate, R-3-hydroxybutyrate-R-1,3-butanediol monoester and D-β-hydroxybutyrate-(R)-1,3-butanediol. Oral administration of such compounds and determination of plasma levels of resulting ketone bodies for other purposes have previously been described. See e.g., Hashim and Vanitallie, J. Lipid Res. 55: 1818-1826 (2014) and Kashiwaya et al., J. Biol. Chem. 285: 25950-29956 (2010).

For purposes of the invention, the term “in combination with” means administration in any order, including simultaneous administration, as well as temporally spaced order from a few seconds up to several days apart.

### References

Buse, D. C. and Lipton, R. B. (2013) Global perspectives on the burden of episodic and chronic migraine. Cephalalgia 33, 885-90

Stovner, L. J. et al. (2014) Neurological disorders in the Global Burden of Disease 2010 study. Acta Neurol. Scand. 129, 1-6

Yun-Hee Youm et al., The ketone metabolite β-hydroxybutyrate blocks NLRP3 inflammasome-mediated inflammatory disease. Nature Medicine 21, 263-269 (2015) doi:10.1038/nm.3804

Christian Waeber, Michael A. Moskowitz, Migraine as an inflammatory disorder. Neurology May 24, 2005 vol. 64 no. 10 suppl 2 S9-S15

Brietzke E, Mansur R B, Grassi-Oliveira R, Soczynska J K, McIntyre R S. Inflammatory cytokines as an underlying mechanism of the comorbidity between bipolar disorder and migraine. Med Hypotheses. 2012 May;78(5):601-5. doi: 10.1016/j.mehy.2012.01.036. Epub 2012 Feb 25.

Hashim and Vanitallie, (2014) Ketone Body Therapy: From the ketogenic diet to the oral administration of ketone ester. J. Lipid Res. 55: 1818-1826.

Castellano C A , Cunnane S C, et al., Lower Brain 18F-Fluorodeoxyglucose Uptake But Normal 11C-Acetoacetate Metabolism in Mild Alzheimer's Disease Dementia. J Alzheimers Dis. 2014 Aug 21.

Pietrobon, D. and Moskowitz, M. a (2013) Pathophysiology of migraine. Annu. Rev. Physiol. 75, 365-91

Yorns, W. R. and Hardison, H. H. (2013) Mitochondrial dysfunction in migraine. Semin. Pediatr. Neurol. 20, 188-93

Courchesne-Loyer, A. et al. (2013) Stimulation of mild, sustained ketonemia by medium-chain triacylglycerols in healthy humans: Estimated potential contribution to brain energy metabolism. Nutrition 29, 635-640

Strahlman, R. S. (2006) Can ketosis help migraine sufferers? A case report. Headache 46, 182

Maggioni, F. et al. (2011) Ketogenic diet in migraine treatment: a brief but ancient history. Cephalalgia 31, 1150-1

Di Lorenzo C, Coppola G, Sirianni G, Di Lorenzo G, Bracaglia M, Di Lenola D, Siracusano A, Rossi P, Pierelli F. Migraine improvement during short lasting ketogenesis: a proof-of-concept study. Eur J Neurol. 2015 Jan;22(1):170-7. doi: 10.1111/ene.12550. Epub 2014 Aug 25.

Christine Marie, Anne-Marie Bralet, and Jean Bralet Laboratoire de Pharmacodynamie et Physiologie Pharmaceutique, Faculte de Pharmacie, Universite de Dijon, Dijon, France, Protective Action of 1,3-Butanediol in Cerebral Ischemia. A Neurologic, Histologic, and Metabolic Study Journal of Cerebral Blood Flow and Metabolism 7:794-800

Kashiwaya et al., (2010) A ketone ester diet increases brain malonyl-CoA and uncoupling proteins 4 and 5 while decreasing food intake in the normal Wistar rat. J. Biol. Chem. 285: 25950-29956.

Veech, R. L., Valeri, C. R. and Vanitallie, T. B. (2012) The mitochondrial permeability transition pore provides a key to the diagnosis and treatment of traumatic brain injury. IUBMB Life 64: 203-207.

Prins, M. (2008) Diet, ketones and neurotrauma. Epilepsia 48(Suppl 8): 111-113.

Fernando W M, Martins R N, et al., The role of dietary coconut for the prevention and treatment of Alzheimer's disease: potential mechanisms of action. Br J Nutr. 2015 May 22:1-14.

Saatman et al., (2008) Classification of traumatic brain injury for Targeted therapies. J. Neurotrauma 25:719-738.

Prins, M., Greco, T., Alexander, D and Giza, C. C. (2013) The pathophysiology of traumatic brain injury at a glance. Disease Models and Mechanisms 6: 1307-1315.

Gaisor, M., Rogawski, M. A. and Hartman, A. L., (2006) Neuroprotective and disease-modifying effects of the ketogenic diet. Behav. Pharmacol. 17: 431-439.

Prins, M. L. and Matsumoto, J. (2014) The collective therapeutic potential of cerebral ketone metabolism in traumatic brain injury. J. Lipid Res. Doi.10/194/j1r.R046706

Uzar E, Evliyaoglu O, Yucel Y, Ugur Cevik M, Acar A, Guzel I, Islamoglu Y, Colpan L, Tasdemir N. Serum cytokine and pro-brain natriuretic peptide (BNP) levels in patients with migraine. Eur Rev Med Pharmacol Sci. 2011 Oct;15(10):1111-6.

Durham P, Papapetropoulos S. Biomarkers associated with migraine and their potential role in migraine management. Headache. 2013 Sep;53(8):1262-77. doi: 10.1111/head.12174. Epub 2013 Jul 12.

Lippi G, Mattiuzzi C, Cervellin G. C-reactive protein and migraine. Facts or speculations? Clin Chem Lab Med. 2014 Sep;52(9):1265-72. doi: 10.1515/cclm-2014-0011.

Filipović B, Matak I, Lacković Z. Dural neurogenic inflammation induced by neuropathic pain is specific to cranial region. J Neural Transm. 2014 May;121(5):555-63. doi: 10.1007/s00702-013-1144-4. Epub 2013 Dec 24.

Welch K M, Brandes A W, Salerno L, Brandes J L.C-reactive protein may be increased in migraine patients who present with complex clinical features. Headache. 2006 Feb;46(2):197-9.

Tajti J, Szok D, Majláth Z, Tuka B, Csáti A, Vécsei L. Migraine and neuropeptides. Neuropeptides. 2015 Mar 30. pii: S0143-4179(15)00021-9. doi: 10.1016/j.npep.2015.03.006.

What is claimed is:

1. A method for preventing or reducing the frequency of migraine onset in a migraineur comprising providing the migraineur with dietary KMCT.

2. The method according to claim 1, wherein the KMCT comprise a mixture of capric and caprylic triglycerides.

3. The method according to claim 1, wherein the KMCT are enriched or purified from coconut oil.

4. The method according to claim 1, wherein the KMCT comprise a mixture of capric and caprylic triglycerides and coconut oil.

5. The method according to claim 1, wherein the total KMCT provided to the migraineur is from about 3g to about 30 g/day.

6. The method according to claim 5, wherein the total KMCT provided to the migraineur is from about 3 g to about 15 g/day.

7. A method for reducing the migraine symptoms experienced by a migraineur comprising providing the migraineur with dietary KMCT.

8. The method according to claim 7, wherein the KMCT comprise a mixture of capric and caprylic triglycerides.

9. The method according to claim 7, wherein the KMCT are enriched or purified from coconut oil.

10. The method according to claim 7, wherein the KMCT comprise a mixture of capric and caprylic triglycerides and coconut oil.

11. The method according to claim 7, wherein the total KMCT provided to the migraineur is from about 3 g to about 30 g/day.

12. The method according to claim 11, wherein the total KMCT provided to the migraineur is from about 10 g to about 30 g/day.

13. A method for augmenting the effects of pharmaceutical intervention to reduce the frequency of migraine onset in a migraineur comprising providing the migraineur with dietary KMCT in combination with a pharmaceutical agent provided to reduce the frequency of migraine onset in a migraineur.

14. The method according to claim 13, wherein the KMCT comprise a mixture of capric and caprylic triglycerides.

15. The method according to claim 13, wherein the KMCT are enriched or purified from coconut oil.

16. The method according to claim 13, wherein the KMCT comprise a mixture of capric and caprylic triglycerides and coconut oil.

17. The method according to claim 13, wherein the total KMCT provided to the migraineur is from about 3 g to about 30 g/day.

18. The method according to claim 17, wherein the total KMCT provided to the migraineur is from about 3 g to about 15 g/day.

19. The method according to claim 13, wherein the pharmaceutical agent is selected from one or more of beta blockers, calcium channel blockers, tricyclic antidepressants, anticonvulsants (including Depakote and Topamax), and Botox.

20. A method for augmenting the effects of pharmaceutical intervention to reduce the migraine symptoms experienced by a migraineur comprising providing the migraineur with dietary KMCT in combination with a pharmaceutical agent provided to reduce migraine symtoms experienced by a migraineur.

21. The method according to claim 20, wherein the KMCT comprise a mixture of capric and caprylic triglycerides.

22. The method according to claim 20, wherein the KMCT are enriched or purified from coconut oil.

23. The method according to claim 20, wherein the KMCT comprise a mixture of capric and caprylic triglycerides and coconut oil.

24. The method according to claim 20, wherein the total KMCT provided to the migraineur is from about 3 g to about 30 g/day.

25. The method according to claim 24, wherein the total KMCT provided to the migraineur is from about 10 g to about 30 g/day.

26. The method according to claim 20, wherein the pharmaceutical agent is selected from one or more of aspirin, acetominaphen, non-steroidal anti-inflammatory drugs (NSAIDs, including ibuprofen and naproxofen), triptans (including sumatriptan (Imitrex), rizatriptan (Maxalt), almotriptan (Axert), naratriptan (Amerge), zolmitriptan (Zomig), frovatriptan (Frova) and eletriptan (Relpax)), ergotamines (including Ergotamine and Dihydroergotamine), opioid medications, and glucocorticoids (including prednisone and dexamethasone).

27. A method for augmenting the effects of non-pharmaceutical intervention to reduce the frequency of migraine onset in a migraineur comprising providing the migraineur with dietary KMCT in combination with a non-pharmaceutical intervention or agent.

28. The method according to claim 27, wherein the KMCT comprise a mixture of capric and caprylic triglycerides.

29. The method according to claim 27, wherein the KMCT are enriched or purified from coconut oil.

30. The method according to claim 27, wherein the KMCT comprise a mixture of capric and caprylic triglycerides and coconut oil.

31. The method according to claim 27, wherein the total KMCT provided to the migraineur is from about 3 g to about 30 g/day.

32. The method according to claim 31, wherein the total KMCT provided to the migraineur is from about 3 g to about 15 g/day.

33. The method according to claim 27, wherein the non-pharmaceutical intervention or agent is selected from one or more of transcutaneous electrical nerve stimulation, transcranial magnetic stimulation, biofeedback, acupuncture, cognitive behavior therapy, ketogenic dieting, butterbur extracts, feverfew, L-cysteine, riboflavin, coenzyme-Q supplements, ketone esters and/or or ketone salts, and magnesium supplements.

34. A method for augmenting the effects of non-pharmaceutical intervention to reduce migraine symptoms experienced by a migraineur comprising providing the migraineur with dietary KMCT in combination with a non-pharmaceutical intervention or agent.

35. The method according to claim 20, wherein the KMCT comprise a mixture of capric and caprylic triglycerides.

36. The method according to claim 20, wherein the KMCT are enriched or purified from coconut oil.

37. The method according to claim 20, wherein the KMCT comprise a mixture of capric and caprylic triglycerides and coconut oil.

38. The method according to claim 20, wherein the total KMCT provided to the migraineur is from about 3 g to about 30 g/day.

39. The method according to claim 24, wherein the total KMCT provided to the migraineur is from about 10 g to about 30 g/day.

40. The method according to claim 34, wherein the non-pharmaceutical intervention or agent is selected from one or more of caffeine, melatonin, magnesium, feverfew, butterbur, willow extract, ginger, valerian, coriander seed, dong quai root, rosemary, linden, honeysuckle, mullien, yarrow, wintergreen and evadia.

41. A method for preventing or reducing the frequency of onset of migraine, and/or reducing migraine symptoms, comprising providing a migraineur with dietary ketone esters and/or or ketone salts in an amount sufficient to raise plasma ketone bodies to a level from about 2 mM to about 5 mM.

42. The method according to claim 41, wherein the ketone ester or ketone salt is selected from one or more of β-hydroxybutyrate-1,3-butanediol monoester, glyceryl-tris-3-hydroxybutyrate, R-3-hydroxybutyrate-R-1,3-butanediol monoester and D-β-hydroxybutyrate-(R)-1,3-butanediol.
